(19) United States
(12) Patent Application Publication
(10) Pub. No.: US 2011/0034776 A1
(43) Pub. Date: Feb. 10, 2011

(54) VAGINAL SPECULUM

(76) Inventors: Richard Albert Dixon, Bountiful, UT (US); Fidelma Kavanagh, Dublin (IE); Raymond Joseph O'Sullivan, Kilkenny (IE); Walter Prendiville, Bublin (IE); Steve Russell Smith, Draper, UT (US); Ivan Vetcnik, Taylorsville, UT (US); William Dean Wallace, Lehi, UT (US)

Correspondence Address: BIRCH STEWART KOLASCH & BIRCH, PO BOX 747, FALLS CHURCH, VA, 22040-0747, US

(21) Appl. No.: 12/536,314

(22) Filed: Aug. 5, 2009

(51) Int. Cl. A61B 1/32

(52) U.S. Cl. 600/205; 600/235

(57) ABSTRACT

A vaginal speculum adapted to be positioned within a vagina has a hollow cylindrical body with means integral therewith for distending the vagina internally of the introitus, whereby the speculum allows visualisation of, and access to and through the cervix, without distending the cervix. In one embodiment the hollow cylindrical body has an inner and outer tube, which are movable relative to each other along the longitudinal axis of the hollow cylindrical body between a first position, wherein the distending means is closed and a second position, wherein the distending means is distended. In this embodiment the distending means is a plurality of elongate members connected between the distal ends of the inner and outer tubes. In the first position the elongate members lie along the longitudinal axis of the hollow cylindrical and in the second position they are bent out from the hollow cylindrical body as the distance between the distal ends of the inner and outer tubes has been shortened.

### BACKGROUND OF THE INVENTION

1. Field of the Invention

The invention relates to specula, and in particular, to vaginal specula.

2. Background Information

A vaginal speculum is a medical device used for examination of the vagina and cervix and in order to gain access to the uterus via the vagina. The speculum is inserted into the vagina to dilate it for examination of the vagina and cervix and to allow access to the uterus.

Vaginal specula are available in three basic shapes (named for Simms, Pederson and Graves) and are made of metal or plastics. They are available in a number of sizes. The more common types consist of a hollow cylinder with a rounded end that is divided into two hinged parts, somewhat like the beak of a duck. The hinged parts of a Pederson type speculum are smaller and less curved than those of the Graves type. The speculum is inserted through the introitus into the vagina with the hinged parts folded together. Once positioned in the vagina the hinged parts are moved apart by manipulation of a lever attached to one of the hinged parts, so as to dilate the vagina, in order to facilitate access to and examination of the vagina and cervix.

However, when the speculum is correctly positioned the fulcrum of the lever will lie outside the introitus. Thus, the action of levering apart the hinged parts will inevitably lead to the distension of the introitus as well as the vagina. It is only when the speculum is opened that the cervix can be visualized.

A disadvantage of such a speculum is that distending the introitus causes discomfort or pain in the majority of women, especially non-parous women.

U.S. Pat. No. 7,041,056 describes a speculum adapted to be positioned inside an orifice, such as a vagina, and inflated for viewing an inside of the orifice. When used as a vaginal speculum the device is inserted into the vagina in similar fashion to a tampon. However a portion of the device remains protruding from the introitus. Once in place the speculum is inflated and takes on a hollow cylindrical shape, resulting in the distension of the vagina.

However, as the device protrudes from the introitus, inflation of the device causes distension of the introitus as well as the vagina resulting in discomfort for the woman being examined.

It is an object of the present invention to overcome the disadvantages of the specula hereinbefore described.

### SUMMARY OF THE INVENTION

Thus, the invention provides a vaginal speculum comprising a hollow cylindrical body and having means integral therewith for distending the vagina internally of the introitus in use, whereby the speculum allows visualisation of, and access to and through the cervix, without distending the introitus.

The advantage of this speculum is that the introitus is not distended and thus, the speculum is more comfortable for the patient in use.

Preferably, the hollow cylindrical body is partitioned into two or more channels.

This feature allows visibility of the upper vagina and cervix through the device. The channels facilitate the use of various instruments such as illuminating means, cameras, sampling instruments and the like.

Preferably, the channels are selected from the group consisting of light, operative, sampling and visibility channels.

The channels can be specifically adapted for accommodating light instruments, operative instruments, sampling instruments or visibility instruments.

Preferably, the distending means distends the upper third of the vagina, in use.

The advantage of this feature is that distending the upper third of the vagina results in minimal discomfort for a patient. Thus, the device allows distension of the upper third of the vagina which allows visualization of the cervix and access to and through the cervix without distending the introitus.

According to one embodiment of the invention, the distending means elevates the anterior wall of the vagina only.

The advantage of this feature is that some procedures, for example a smear test, may only require the elevation of the anterior wall of the vagina. Traditional vaginal specula distend both the anterior and posterior walls, resulting in greater discomfort.

According to an alternative embodiment, the distending means are disposed circumferentially on the hollow cylindrical tube.

According to a still further embodiment, the distending means are disposed bilaterally on the hollow cylindrical tube.

Preferably, the distending means is a mechanical means.

Where the distending means is a mechanical means, the device is easy to manufacture and use.

Suitably, the mechanical means is a trocar/cannula arrangement.

The advantage of this feature is that the device has a bull-nosed tip so that the device can go very gently through the introitus in use.

According to a further embodiment, the hollow cylindrical body has an inner and outer tube, which are movable relative to each other along the longitudinal axis of the hollow cylindrical body between a first position, wherein the distending means is closed and a second position, wherein the distending means is extended.

An advantage of this arrangement is that movement of the device from the first to the second position and back again is in a straight line and therefore no levering action is involved. This reduces the discomfort to the patient.

Preferably, the distending means is a plurality of elongate members, each elongate member being attached at one end thereof to the distal end of the inner tube and at the other end thereof to the distal end of the outer tube, with the elongate members being disposed around the circumference of the hollow cylindrical body.

The disposition of the elongate members means that when they are extended in the second position they will distend the vagina on all sides.

Further, preferably, each elongate member is parallel to the longitudinal axis of the hollow cylindrical body in the first position, and wherein movement thereof to the second position shortens the distance between the distal ends of the inner tube and results in the bending of each elongate member away from the hollow cylindrical body.

An advantage of this arrangement is that the walls of the vagina will only be distended in the area of the elongate members. Thus, positioning the speculum at various depths within the vagina allows visualisation and possible sampling of the vaginal wall at the various positions.

Alternatively, the distending means is a hydraulic means.

The use of hydraulic means facilitates the smooth extension of the distending means resulting in minimal discomfort to the patient.

In a further embodiment, the distending means is a pneumatic means.

Pneumatic means provide a simple method for distending the vagina in a step-by-step manner.

Preferably, the pneumatic means comprise an inflatable balloon.

In a further embodiment, the hollow cylindrical tube is provided with an external circumferential sheath.

The sheath protects the speculum. Furthermore, it is used to make the speculum more hygienic as the sheath can be disposable.

Preferably, part of the external surface of the hollow cylindrical tube is covered by a sheath.

Suitably, the cervix-engaging end of the sheath is open.

Alternatively, the cervix-engaging end of the sheath opens on distension of the speculum.

This feature allows visualization of the cervix through the sheath. Furthermore, this feature allows access to and through the cervix.

Alternatively, the cervix-engaging end of the sheath is occluded.

Preferably, the cervix-engaging end of the sheath is partially occluded.

Further, preferably, a camera is accommodated within the sheath.

The camera allows visualization of the cervix and the vaginal walls, in use.

### BRIEF DESCRIPTION OF THE DRAWINGS

The invention will be further illustrated by the following description of embodiments thereof, given by way of example only with reference to the accompanying drawings in which:

FIG. 1 is a view of a device according to the invention in a first position;

FIG. 2 is a view of the device of FIG. 1, including a protective cap;

FIG. 3 is a view of the device of FIG. 1 in a second position;

FIG. 4 is a view of a second embodiment of a device according to the invention in a first position; and

FIG. 5 is a view of the embodiment of FIG. 4 in a second position.

### DETAILED DESCRIPTION OF PREFERRED EMBODIMENTS

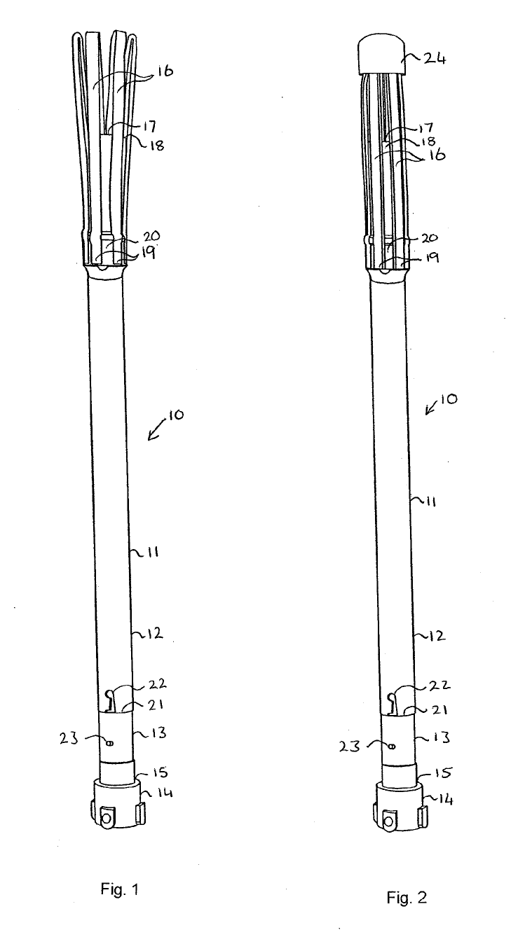

Referring to FIG. 1 there is illustrated, generally at 10, a vaginal speculum, having a hollow cylindrical body 11 consisting of an outer tube 12 and an inner tube 13, slidably mounted within the outer tube 12.

The inner tube 13 has a grip 14 mounted at one end 15 thereof and means for distending the vagina internally of the introitus, in use, consisting of a plurality of elongate members 16 attached by their ends 17 to an inner circumferential surface (not shown) of the other end 18 of the innertube 13.

The other ends 19 of the plurality of elongate members 16 are attached circumferentially around end 20 of the outer tube 12. At the other end 21 of the outer tube 12 there is a retaining slot 22 which cooperates with a pin 23 on the inner tube 13.

Referring to FIG. 2, the vaginal speculum 10 is illustrated with a protective cap 24 in place, which protects the plurality of elongate members 16, when not in use.

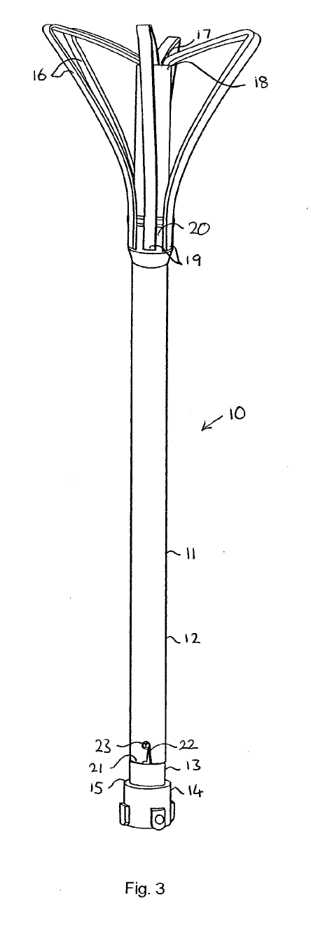

Referring to FIGS. 1 and 3, the vaginal speculum 10 is moved from a first position in FIG. 1 to a second position in FIG. 2 by pushing the inner tube 13 through the outer tube 12, so that the plurality of elongate members 16 are spread outwards from the end 18 of the inner tube 13, as illustrated in FIG. 3. As the inner tube 13 is pushed through the outer tube 12, the pin 23 on the inner tube 13 engages the retaining slot 22. Once the plurality of elongate members 16 is fully extended the grip 14 is turned as illustrated in FIG. 3, such that the pin 23 is held in the retaining slot 22 and the vaginal speculum 10 is maintained in the second position.

In use, the cap 24 is removed from the speculum 10, the speculum 10 is introduced through the introitus of the patient, while maintaining it in the first position as illustrated in FIG. 1. The narrowness of the device prevents distension of the introitus. Once the vaginal speculum 10 is in position the speculum 10 is moved to the second position illustrated in FIG. 3, whereby the plurality of elongate members 16 spread outwards to distend the vagina internally of the introitus. Thus, the vaginal speculum 10 allows visualisation of, and access to and through the cervix, without distension of the introitus.

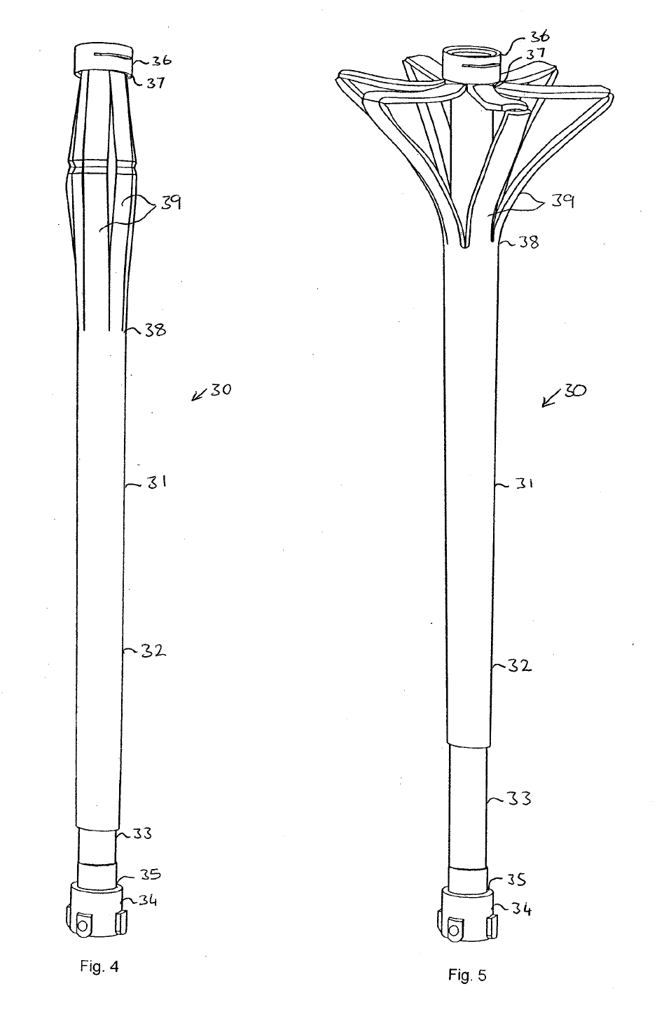

Referring to FIG. 4 there is illustrated, generally at 30, a second embodiment of a vaginal speculum according to the invention, having a hollow cylindrical body 31 consisting of an outer slidable tube 32 and an inner tube 33.

The inner tube 33 has a grip 34 mounted at one end 35 thereof and a collar 36 at the other end 37 thereof. The outer slidable tube 32 divides at one end 38 into a plurality of elongate members 39. The plurality of elongate members 39 are attached to the underside of the collar 36 at the end 37 of the inner tube 33.

Referring to FIGS. 4 and 5, the vaginal speculum 30 is moved from a first position as illustrated in FIG. 4 to a second position as illustrated in FIG. 5, by pushing the outer slidable tube 32 along the inner tube 33, so that the plurality of elongate members 39 spread outwards, as illustrated in FIG. 5. The vaginal speculum 30 has means (not shown) for retaining the vaginal speculum 30 in the second position.

In use, the vaginal speculum 30 (in the first position illustrated in FIG. 4) is passed through the introitus of the patient. The narrowness of the device prevents distension of the introitus. Once the vaginal speculum 30 is in position the speculum 30 is moved into the second position illustrated in FIG. 5, whereby the plurality of elongate members 39 spread outwards to distend the vagina internally of the introitus. Thus, the vaginal speculum 30 allows visualisation of, and access to and through the cervix, without distending the introitus.

1. A vaginal speculum comprising a hollow cylindrical body and having means integral therewith for distending the vagina internally 5 of the introitus in use, whereby the speculum allows visualisation of, and
access to and through the cervix, without distending the introitus.

2. A speculum according to claim 1, wherein the hollow cylindrical body is partitioned into two or more channels.

3. A speculum according to claim 2, wherein the channels are 10 selected from the group consisting of light, operative, sampling and visibility channels.

4. A speculum according to claim 1, wherein the distending means distends the upper third of the vagina, in use.

5. A speculum according to claim 1, wherein the 15 distending means elevates the anterior wall of the vagina only.

6. A speculum according to claim 1, wherein the distending means are disposed circumferentially on the hollow cylindrical body.

7. A speculum according to claim 1, wherein the 20 distending means are disposed bilaterally on the hollow cylindrical body.

8. A speculum according to claim 1, wherein the distending means is a mechanical means.

9. A speculum according to claim 8, wherein the mechanical means is a trocar/cannula arrangement.

10. A speculum according to claim 8, wherein the hollow cylindrical body has an inner and outer tube, which are movable relative to each other along the longitudinal axis of the hollow cylindrical body between a first position, wherein the distending means is closed and a second position, wherein the distending means is extended.

11. A speculum according to claim 1, wherein the distending means is a plurality of elongate members, each elongate member being attached at one end thereof to the distal end of the inner tube and at the other end thereof to the distal end of the outer tube, with the elongate members being disposed around the circumference of the hollow cylindrical body.

12. A speculum according to claim 11, wherein each elongate member is parallel to the longitudinal axis of the hollow cylindrical body in the first position, and wherein movement thereof to the second position shortens the distance between the distal ends of the inner tube and results in the bending of each elongate member away from the hollow cylindrical body.

13. A speculum according to claim 1, wherein the distending means is a hydraulic means.

14. A speculum according to claim 1, wherein the distending means is a pneumatic means.

15. A speculum according to claim 14, wherein the pneumatic means comprises an inflatable balloon.

16. A speculum according to claim 1, wherein the hollow cylindrical body is provided with an external circumferential sheath.

17. A speculum according to claim 1, wherein part of the external surface of the hollow cylindrical body is covered by a sheath.

18. A speculum according to claim 1, wherein the cervix-engaging end of the sheath is open.

19. A speculum according to claim 1, wherein the cervix-engaging end of the sheath is occluded.

20. A speculum according to claim 1, wherein the cervix-engaging end of the sheath is partially occluded.

21. A speculum according to claim 18, wherein a camera is accommodated within the sheath.
